(19) United States
(12) Patent Application Publication
(10) Pub. No.: US 2023/0233352 A1
(43) Pub. Date: Jul. 27, 2023

(54) ORTHOSIS FOR A LOWER EXTREMITY OF A WEARER

(71) Applicant: CO12 GmbH, Lichtenfels (DE)

(72) Inventors: Ingo Güttler, Lichtenfels (DE); Jens Henzler, Lichtenfels (DE); Matthias Mitschke, Güterloh (DE); Theodor Schraeder, Lippe Detmold (DE); Tommy Schafran, Unna (DE)

(73) Assignee: CO12 GmbH, Lichtenfels (DE)

(21) Appl. No.: 17/928,772

(22) PCT Filed: May 31, 2021
(86) PCT No.: PCT/EP2021/064545

§ 371 Date: Nov. 30, 2022

(30) Foreign Application Priority Data
Jun. 5, 2020 (DE) 10 2020 115 001.6

(51) Int. Cl. A61F 5/01; B33Y 80/00; B33Y 50/00

(52) U.S. Cl. CPC A61F 5/0111; B33Y 80/00; B33Y 50/00

(57) ABSTRACT

An orthosis (1) for a lower extremity of a wearer, in particular for a foot and a lower leg of a wearer, preferably for wearers with diabetic foot syndrome or a neuropathic osteoarthropathy relating to the foot, comprising:
a first orthosis part (2) which is to be arranged or which can be arranged on a lower leg of a wearer,
a second orthosis part (3) which is to be arranged or which can be arranged on a foot of a wearer, and
a third orthosis part (4) which connects the first and second orthosis part to one another, wherein
   at least one of the orthosis parts (2-4) has a geometric-constructive design, at least in portions, possibly completely, which is individually configured on the basis of data describing a foot and/or a lower leg of a wearer.

The invention relates to an orthosis for a lower extremity of a wearer, in particular for a foot and a lower leg of a wearer, preferably for wearers with diabetic foot syndrome or a neuropathic osteoarthropathy relating to the foot.

Corresponding orthoses for lower extremities or limbs of a wearer are well-known from the prior art in different embodiments and for different medical fields of application.

An exemplary field of application of corresponding orthoses is the field of technical orthopedics, in which corresponding orthoses are used, for example, for the treatment of diabetic foot syndrome, which is a syndrome of pathological changes based on a painless sensory neuropathy and/or a peripheral arterial occlusive disease in diabetes mellitus, or for the treatment of neuropathic osteoarthropathy relating to the foot.

The orthosis designs known from the prior art, although basically sound, are in need of improvement with regard to functional, constructive and manufacturing aspects. This is especially true against the background of the need for the highest possible degree of individualization of the orthoses, which is expected to have various advantages, such as special medical efficacy, improved compliance, etc.

Based on this, the object underlying the invention is to provide an improved orthosis for a lower extremity of a wearer.

The object is achieved by an orthosis for a lower extremity of a wearer according to claim 1. The claims dependent thereon relate to possible embodiments of the orthosis.

A first aspect of the invention described herein relates to an orthosis for a lower extremity of a wearer, in particular for a foot and a lower leg of a wearer, preferably for a wearer with diabetic foot syndrome or a neuropathic osteoarthropathy relating to the foot. In the following, the term “wearer” is used, which includes both male and female wearers. The same applies to the term “patient”.

In general, the orthosis is typically a component or group of components belonging to the field of technical orthopedics. The orthosis is typically used in particular for the treatment of diabetic foot syndrome or a neuropathic osteoarthropathy relating to the foot and the associated multitude of concomitant and secondary diseases. The orthosis is thus particularly configured for the treatment of diabetic foot syndrome or a neuropathic osteoarthropathy relating to the foot and the associated multitude of concomitant and secondary diseases. The orthosis is thus intended in particular for wearers or patients with diabetic foot syndrome or a neuropathic osteoarthropathy relating to the foot and the associated multitude of concomitant and secondary diseases.

In principle, however, the orthosis can be used for stabilization and immobilization in the case of diseases of the extrinsic foot musculature, such as tibialis posterior dysfunction or tibialis anterior syndrome. Furthermore, the orthosis can also be suitable for early mobilization of a patient, for example after an operation on a lower extremity. Due to the stabilizing character of the orthosis and a patient-specific shape, early mobilization can be possible in the context of early rehabilitation.

The orthosis typically comprises three orthosis parts, the functional and constructive design of which is explained in greater detail below. The orthosis is thus constructed in multiple parts, i.e., as will be seen in the following, in particular in three parts, or in a modular fashion.

The orthosis comprises a first orthosis part which can be arranged or which is to be arranged on a lower leg, i.e. in particular on or in the region of a shin of a wearer, i.e. in particular in the frontal region just below the patella of a wearer. The first orthosis part, which may also be referred to as the first orthosis component, is thus configured with regard to its functional and constructive design in such a way that it can be arranged on a lower leg of a wearer or is arranged on a lower leg of the wearer when the orthosis is worn as intended. The first orthosis part thus extends at least in portions along and/or around a lower leg of a wearer when the orthosis is worn as intended. The first orthosis part can enclose the lower leg of a wearer at least in portions, possibly completely. The first orthosis part thus has one or more contact regions or areas which lie against the lower leg of a wearer when the orthosis is worn as intended. The one or more contact regions or areas can surround the lower leg of a wearer, in particular in a circumferential direction, at least in portions, in particular in a form-fitting manner. The dimensions of the first orthosis part can be adapted to the dimensions, i.e. in particular width, length, circumference, etc., of the lower leg. The first orthosis part may also be designated or considered as a lower leg part, as appropriate.

The orthosis further comprises a second orthosis part that can be arranged or which is to be arranged on a foot of a wearer. The second orthosis part, which may also be referred to as a second orthosis component, is therefore configured with regard to its functional and constructive design in such a way that it can be arranged on a foot of a wearer or is arranged on a foot of the wearer when the orthosis is worn as intended. The second orthosis part thus extends at least in portions along and/or around a foot of a wearer when the orthosis is worn as intended. The second orthosis part can enclose the foot of a wearer at least in portions, possibly completely. The second orthosis part thus has one or more contact regions or areas which lie against the foot of a wearer when the orthosis is worn as intended. The dimensions of the second orthosis part can be adapted to the dimensions of the foot, i.e. in particular width, length, circumference, etc. The second orthosis part may also be designated or considered as a foot part, as appropriate.

The orthosis further comprises a third orthosis part connecting the first and the second orthosis part to one another. The third orthosis part, which may also be referred to as a third orthosis component, is therefore configured with regard to its functional and constructive design in such a way that it can be arranged at least in portions between the first and second orthosis parts in order to connect the first orthosis part to the second orthosis part. With regard to its functional as well as constructive design, the third orthosis part is therefore configured in such a way that it comprises at least one first interface via which it can be fastened to the first orthosis part, i.e. in particular to at least one interface provided for this purpose, possibly a corresponding interface, and comprises at least one second interface via which it can be fastened to the second orthosis part, i.e. in particular to at least one interface provided for this purpose, possibly a corresponding interface. In the intended worn state of the orthosis, the third orthosis part typically extends in a distal or proximal direction relative to the leg of the wearer between the first orthosis part and the second orthosis part. The third orthosis part can be arranged medially or laterally on the leg of a wearer when the orthosis is worn as intended. An at least partially dorsal arrangement of the third orthosis part is also conceivable. The third orthosis part may also be referred to or considered to be a connecting part, as appropriate.

The orthosis parts are therefore set up via respective interfaces to cooperate with each other to form the orthosis. For this purpose, the orthosis parts can in particular be fastened to or are fastened to each other. In particular, the first orthosis part can be fastened to the second orthosis part via the third orthosis part, and vice versa. The respective interfaces of the first orthosis part and/or of the second orthosis part and/or of the third orthosis part can be configured as mechanical interfaces which are set up to cooperate mechanically in order to fasten the respective orthosis parts on or to each other.

The third orthosis part can be fastened to the first orthosis part and/or to the second orthosis part, for example, by means of form-fitting and/or frictionally engaging fastening types. For example, clamping and/or detent/snap-in and/or screw and/or plug-in and/or tensioning fastenings can be used as fastening types and thus clamping and/or detent/snap-in and/or screw and/or plug-in and/or tensioning elements that implement corresponding clamping and/or detent/snap-in and/or screw and/or plug-in and/or tensioning fastenings can be used as fastening elements.

In all embodiments, the fastening of the third orthosis part to the first orthosis part and/or to the second orthosis part can be detachable (without damage or destruction).

The fastening of the third orthosis part to the first orthosis part and/or to the second orthosis part can therefore be cancelled and restored, if necessary, which has advantages with regard to possible adjustments, repairs, etc. of the orthosis or the orthosis parts.

In addition to corresponding interfaces which allow the various orthosis parts to be fastened on or to each other, positioning devices can also be formed on one or more orthosis parts and allow the orthosis parts that are to be fastened to each other to be positioned or oriented exactly as one—this may allow pre-assembly positions of the orthosis parts to be realized.

For example, at least one corresponding positioning device can be arranged or formed on the first orthosis part and allows exact positioning of the third orthosis part to be fastened on or to the first orthosis part. A corresponding positioning device can be, for example, a recess or depression in the first orthosis part, at or in which the corresponding portion of the third orthosis part can be inserted. A corresponding recess or depression can be geometrically configured in such a way that it has a contouring that is the opposite of the contour of the corresponding portion of the third orthosis part. A reversed configuration is also conceivable. The same can be achieved with other measures that have the same effect, for example with interacting plug-in elements.

Alternatively or additionally, at least one corresponding positioning device can be arranged or formed on the second orthosis part and allows an exact positioning of the third orthosis part to be fastened on or to the second orthosis part. A corresponding positioning device can be, for example, a recess or depression in the second orthosis part, at or in which the corresponding portion of the third orthosis part can be inserted. A corresponding recess or depression can be configured in a geometric-constructive respect in such a way that it has a contouring that is the opposite of the contour of the corresponding portion of the third orthosis part. Again, a reverse configuration is equally conceivable. The same can be achieved with other measures that have the same effect, for example with interacting plug-in elements.

In principle, however, the term “orthosis” as used herein refers to both an orthosis in its intended mounted state, in which the first and second orthosis parts are fastened or connected to each other via the third orthosis part, and in its unmounted state, in which the first and second orthosis parts are not fastened or connected to each other via the third orthosis part.

At least one of the orthosis parts, i.e. in particular the first orthosis part and/or the second orthosis part, preferably both the first orthosis part and the second orthosis part, has a geometric-constructive design, at least in portions, possibly completely, which is individually configured on the basis of data describing a foot and/or a lower leg of a wearer. At least one orthosis part thus has an individually configured geometric-constructive design and thus a high degree of individualization. In particular, a respective orthosis part may be an individually configurable or individually configured individual part for a specific limb, i.e. in particular a specific foot or a specific lower leg, of a specific wearer. The orthosis part can represent an individually configured (negative) image of a lower leg or foot portion, on which the orthosis part is to be arranged, of a specific wearer. This allows an individually configured and thus exact fit of the orthosis part specific to the wearer or the wearer's body part.

The individually configured geometric-constructive design of an orthosis part—this can be understood in particular to mean a specific contouring of the orthosis part—was generated, as mentioned, on the basis of data describing a foot and/or a lower leg of a wearer at least in portions, possibly completely. Corresponding data can, for example, be acquisition data that can be acquired or are acquired via a suitable acquisition device. A corresponding acquisition device may, for example, be or comprise an optical acquisition device, such as an image acquisition device, i.e., for example, a camera or scanning device. Corresponding data can therefore be, for example, one-dimensional or multi-dimensional image data, i.e. in particular camera, scan, CT or MRT data, of a foot and/or a lower leg of a wearer.

Various advantages result from the individually configured geometric-constructive design of the one or more orthosis parts of the orthosis. First of all, the individually configured geometric-constructive design of one or more orthosis parts of the orthosis has a positive effect on the wearing properties of the orthosis. In addition, the multi-part or modular structure of the orthosis offers a targeted, need-based, in particular weight-optimized, stiffening option for the orthosis parts in order to immobilize the foot or leg based on the disease.

A particularly expedient embodiment of the orthosis provides that both the first orthosis part has a geometric-constructive design which is individually configured at least in portions, possibly completely, on the basis of data describing a lower leg of a wearer, and the second orthosis part has a geometric-constructive design which is individually configured at least in portions, possibly completely, on the basis of data describing a foot of a wearer. Alternatively or additionally, the third orthosis part can also have a geometric-constructive design that is individually configured on the basis of data describing, for example, a lower leg of a wearer.

Overall, an improved orthosis for a lower extremity of the wearer is available, especially due to the high degree of individualization.

The individually configured geometric-constructive design of an orthosis part can be expediently realized in terms of production technology, in particular with additive manufacturing processes—sometimes also referred to as “3D printing”. Consequently, at least one orthosis part having an individually configured geometric-constructive design can be additively manufactured at least in portions, if necessary, completely. Likewise, a plurality of orthosis parts, i.e. possibly also all orthosis parts, can be additively manufactured. Additive manufacturing allows components with special structural, i.e. both functional and constructive, properties to be formed, which with conventional manufacturing processes cannot be realized or can only be realized to a limited extent.

In principle, all additive manufacturing processes can be considered. In particular, additive manufacturing processes that can be used to process metals or plastics into a three-dimensional body can be considered. In particular, additive manufacturing processes which allow an additive construction of three-dimensional bodies by layer-by-layer selective consolidation of a layer-by-layer selectively consolidatable, optionally powdery, build material—this may be, for example, a metal or a plastic, such as polyamide—by means of radiant energy, i.e. in particular by means of at least one energy beam, may be considered. In this context, reference is made merely by way of example to multi-jet fusion processes, binder jetting processes, CLIP processes, selective laser fusion processes, selective electron beam fusion processes, selective laser sintering processes, fused deposition modelling (“FDM”) processes, continuous filament fabrication (“CFF”) processes or stereolithography processes, etc. The selection of a suitable additive manufacturing process is typically determined by various requirements of the particular orthosis part, such as material, chemical and/or physical properties, etc.

In all embodiments, the first orthosis part can comprise a main body which has a receiving area, in particular accessible via an access area, for at least partially receiving a lower leg or a lower leg portion of a wearer. The receiving area of the first orthosis part can be provided with corresponding contact regions or areas which, when the orthosis is worn as intended, lie against the lower leg of a wearer or enclose it at least in portions. The access area is selected in such a way that it is as easy as possible to put on and take off the first orthosis part, i.e. in particular as easy as possible to get in and out of the first orthosis part.

The main body of the first orthosis part can have a geometric-constructive design that is individually configured at least in portions, possibly completely, on the basis of data describing a lower leg of a wearer. Specifically, the main body can, for example, have a geometric-constructive design that at least partially surrounds a lower leg of a wearer, in particular on the shin side, in particular just below the patella. The shape of the main body can be ergonomic. The main body can thus have a semi-shell-like or -shaped basic form individually adapted to a lower leg of a wearer. Viewed cross-sectionally, the main body can have a U- or V-shaped basic shape or geometry individually adapted to a lower leg of a wearer. The main body, i.e. in particular the various contact regions or areas of the main body, can embrace the lower leg of a wearer, in particular in the circumferential direction, at least in portions, in particular in a form-fitting manner. In this way, an undesired rotation of the lower leg of the wearer can be prevented or made more difficult, in particular in cooperation with the second orthosis part and/or third orthosis part. In addition, an (extensive) immobilization of the lower leg can be realized in this way, which is typically also accompanied by an immobilization of the foot. A corresponding immobilization typically has a positive effect from a medical point of view, as for example inflammations can be inhibited.

At least one, in particular tab-like or -shaped or eyelet-like or -shaped, interface for fastening a, in particular band-like or -shaped, fixing means for fixing, in particular as required, a lower leg portion of a wearer in the receiving area can be arranged or formed on the main body of the first orthosis part. A corresponding interface can be formed in one piece (integrally) with the main body or as a separate component from the main body, so that it forms an integral or separate component of the main body. In particular, a corresponding interface can be formed integrally with the main body within the scope of additive manufacturing of the main body. The same applies in the case where the main body has a plurality of corresponding interfaces.

Alternatively or additionally, at least one, in particular hole-like or -shaped and/or thread-like or -shaped interface for fastening a fastening means, in particular acting in a form-fitting and/or frictionally engaging manner, for fastening, in particular releasably, the third orthosis part to the first orthosis part can be arranged or formed on the main body of the first orthosis part. A corresponding interface can be formed in one piece (integrally) with the main body so that it forms an integral part of the main body. In particular, a corresponding interface can be formed integrally with the main body within the scope of an additive manufacturing of the main body. The same applies in the case in which the main body has a plurality of corresponding interfaces. Typically, a plurality of corresponding interfaces are arranged or formed on the first orthosis part, in particular in a defined arrangement. The arrangement of the interfaces is selected in such a way that it allows a desired arrangement and orientation of the third orthosis part on or relative to the first orthosis part.

In all embodiments, the second orthosis part can comprise a main body which has a receiving area, in particular accessible via an access area, for at least partially receiving a foot or a foot portion of a wearer. The receiving area of the second orthosis part can be provided with corresponding contact regions or areas which, when the orthosis is worn as intended, lie against or at least partially enclose the foot of a wearer. The access area is selected in such a way that it is as easy as possible to put on and take off the second orthosis part, i.e. in particular as easy as possible to get in and out of the second orthosis part.

The main body of the second orthosis part can have a geometric-constructive design which is individually configured at least in portions, if necessary, completely, on the basis of data describing a foot of a wearer. Specifically, the main body can, for example, have a geometric-constructive design that surrounds a foot of a wearer, in particular on the sole side and/or (outer and/or inner) instep side and/or tension side, at least in portions. The shape of the main body can be ergonomic. The main body can therefore have, for example, a shoe-like basic shape or geometry individually adapted to a foot of a wearer. In particular, the main body can have a basic shape or geometry of an orthopedic shoe, in particular a shoe for “diabetic feet”, which is individually adapted at least in portions to a foot of a wearer. This can also be understood to mean the formation of a footbed that is individually adapted to the foot of a wearer, at least in portions. The main body of the second orthosis part can therefore have an orthopedic inlay that is individually adapted to the foot of a wearer, at least in portions. A corresponding orthopedic inlay can be designed in particular with regard to a special, i.e. in particular as homogeneous as possible, pressure distribution on the sole of the foot of a wearer. A corresponding orthopedic inlay can, for example, be formed by or comprise elevations and/or depressions that model the sole of a wearer's foot.

At least one, in particular tab-like or -shaped or eyelet-like or -shaped, interface for fastening a, in particular band-like or -shaped, fixing means for fixing, in particular as required, a foot portion of a wearer in the receiving area can be arranged or formed on the main body of the second orthosis part. A corresponding interface can be formed in one piece (integrally) with the main body or as a separate component from the main body, so that it forms an integral or separate component of the main body. In particular, a corresponding interface can be formed integrally with the main body within the scope of additive manufacturing of the main body. The same applies in the case in which the main body has a plurality of corresponding interfaces.

Alternatively or additionally, at least one, in particular hole-like or -shaped and/or thread-like or -shaped interface for fastening a fastening means, in particular acting as a positive or non-positive engagement, for fastening, in particular releasably, the third orthosis part to the first orthosis part can be arranged or formed on the main body of the second orthosis part. A corresponding interface can be formed in one piece (integrally) with the main body so that it forms an integral part of the main body. In particular, a corresponding interface can be formed integrally with the main body within the scope of an additive manufacturing of the main body. The same applies in the case where the main body has a plurality of corresponding interfaces. Typically, a plurality of corresponding interfaces are arranged or formed on the second orthosis part, in particular in a defined arrangement. The arrangement of the interfaces is selected in such a way that it allows a desired arrangement and orientation of the third orthosis part on or relative to the second orthosis part.

The third orthosis part can have an elongate, in particular rail-like or -shaped or strut-like or -shaped, geometric-constructive main body. The main body extends between the first and second orthosis parts when the orthosis is worn as intended. This typically results in a monolateral or monomedial arrangement of the third orthosis part, as the main body only extends along one side of the lower leg of the wearer when the orthosis is worn as intended. As mentioned, the third orthosis part can be arranged medially or laterally on the leg of a wearer when the orthosis is worn as intended. An at least partially dorsal arrangement of the third orthosis part is also conceivable. This simplifies the construction of the orthosis and also has advantages for putting the orthosis on and taking it off.

If the main body is not provided with desired structural properties, i.e. in particular a desired stiffness, due to its geometric-constructive design, the third orthosis part can comprise one or more stiffening elements extending in the longitudinal direction. Corresponding stiffening elements may, for example, have a round, circular or angular cross-sectional geometry. Cross-sectional geometries of a stiffening element that vary in portions are conceivable. In the case of a plurality of stiffening elements, which can basically be present in parallel and/or serial arrangement, stiffening elements can be present with cross-sectional geometries that differ at least in portions. In all embodiments, corresponding stiffening elements can be made of a mechanically rigid material, such as metal, composite material, in particular glass or carbon fiber material, or a corresponding material structure, or may comprise such a material structure.

Corresponding stiffening elements can be designed analogously to the third orthosis part, for example strut-like or -shaped.

The third orthosis part may be strut-like or -shaped, as mentioned. The third orthosis part can therefore be formed by one or more strut elements or can comprise one or more strut elements.

The first orthosis part and the second orthosis part can each comprise at least one receiving or fastening area for receiving or fastening at least one strut element in a form-fitting and/or frictionally engaging and/or integrally bonded manner, i.e. in particular for receiving or fastening a receiving or fastening portion of a strut element, which cooperates with a corresponding receiving or fastening area, on the relevant orthosis part. Corresponding receiving or fastening areas can be formed, for example, by plug-in receptacles. Alternatively or additionally, clamping, tensioning or detent receptacles are conceivable. Alternatively or additionally, adhesion or weld receptacles are conceivable; a corresponding strut element can thus be adhered or welded to a corresponding receptacle or fastening area, for example by means of a single-component or multi-component adhesive. Basically, orthosis-part-side receiving or fastening areas allow a detachable or non-detachable receiving or fastening of a strut element to a corresponding orthosis part.

Corresponding strut elements can also serve as stiffening elements due to their structural properties, i.e. in particular their stiffness. The structural properties, i.e. in particular the stiffness, of the strut elements can result from the material forming the strut elements or the material structure forming the strut elements. For example, the strut elements can be made of metal, fiber-reinforced plastic, fiber composite materials, i.e. in particular glass or carbon fiber composite materials.

Exemplary configurations of the orthosis with at least one strut-like or strut-shaped third orthosis part, i.e. with a third orthosis part which is formed by or comprises one or more strut elements, are explained in more detail below. In principle, it is also true here that corresponding strut elements can be arranged or are arranged typically medially or laterally, i.e. generally to the side, on the leg of a wearer when the orthosis is worn as intended. This makes it easier for a user to get into the orthosis.

A first exemplary configuration provides that at least one strut element is arranged extending medially or laterally between the first and the second orthosis part when the orthosis is worn as intended. The at least one strut element is typically fastened with a first receiving or fastening portion to or in a receiving or fastening area of the first orthosis part and with a second receiving or fastening portion to or in a receiving or fastening area of the second orthosis part.

Another exemplary configuration provides that a plurality of strut elements are arranged extending medially and/or laterally between the first and the second orthosis part when the orthosis is worn as intended. The brace elements, which are oriented for example parallel or at an angle to one another, are each typically fastened with a first receiving or fastening portion to or in a receiving or fastening area of the first orthosis part and with a second receiving or fastening portion to or in a receiving or fastening area of the second orthosis part.

A further exemplary configuration can thus specifically provide that at least one first strut element is arranged extending medially (first side) between the first and the second orthosis part when the orthosis is worn as intended and at least one further strut element is arranged extending laterally (second side) between the first and the second orthosis part when the orthosis is worn as intended. Thus, at least one strut element can be present both laterally and medially, so that strut elements arranged or oriented opposite each other are present. The at least one first orthosis part is typically fastened with a first receiving or fastening portion to or in a medially oriented receiving or fastening region of the first orthosis part and with a second receiving or fastening portion to or in a medially oriented receiving or fastening region of the second orthosis part. The at least one further orthosis part is typically fastened with a first receiving or fastening portion to or in a laterally oriented receiving or fastening area of the first orthosis part and with a second receiving or fastening portion to or in a laterally oriented receiving or fastening area of the second orthosis part.

In particular, an exemplary configuration specifically provides that a plurality of first strut elements are arranged extending medially between the first and the second orthosis part when the orthosis is worn as intended and a (single) strut element is arranged extending laterally between the first and the second orthosis part when the orthosis is worn as intended. A reverse configuration is also conceivable.

For all configurations, it is true in principle that individual, a plurality of, or all first strut elements can be arranged or oriented extending in a first spatial direction. Similarly, individual, a plurality of, or all further strut elements can be arranged or oriented in a spatial direction different from the first spatial direction. The choice of different spatial directions in which the first or further strut elements extend can also have a targeted, i.e. in particular user-specific, influence on the structural properties of the orthosis, for example in order to achieve the best possible effect of the orthosis.

The structural properties of the orthosis can be adjusted or changed in a targeted manner, i.e. in particular user-specifically, via the number or arrangement or orientation of strut elements, for example in order to achieve the best possible effect of the orthosis. In this context, it may be expedient to provide a plurality of receiving or fastening areas on the first orthosis part and/or on the second orthosis part, for example medially and/or laterally, so that the first and/or the second orthosis part, in particular medially and/or laterally, is basically prepared for a plurality of strut elements.

In all configurations with a plurality of strut elements, strut elements can be used which differ in their structural properties, i.e. in particular in their stiffness. First strut elements can therefore have different structural properties, i.e. in particular different stiffness, than other strut elements. Of course, in the case of a plurality of first strut elements, these can also differ in their structural properties. The same applies to the case of a plurality of further strut elements.

The orthosis may generally comprise at least one stabilizing element, i.e. in particular an ankle and/or lower leg stabilizing element, for stabilizing an ankle region and/or a lower leg of a user. A corresponding ankle stabilizing element may, for example, be arranged or formed on strut elements. In particular, a corresponding ankle stabilizing element may be penetrated by strut elements, which ensures a particularly stable arrangement. Specifically, a corresponding stabilizing element can be formed from a plurality of, for example half-shell-like, stabilizing element segments, which are connected to each other in a form-fitting and/or frictionally engaging and/or integrally bonded manner.

At least one interface can also be arranged or formed on a stabilizing element for fastening a fixing means, in particular a band-like or -shaped fixing means. A fixing means fastened to a corresponding interface can, if necessary, additionally allow stabilization of the ankle and/or lower leg of a user.

In conjunction with the third orthosis part, it is generally true that it can have a special mechanical stability, i.e. in particular a special strength and rigidity. The third orthosis part can be designed in such a way that it meets all relevant mechanical requirements. The other orthosis parts can then be configured in a correspondingly more individual way. This applies in particular to a configuration of the orthosis in which the third orthosis part is conventionally manufactured, for example by a conventional molding or shaping process, and the other orthosis parts are additively manufactured.

In principle, it is true for all orthosis parts that they can be configured with or can include functional structures, such as lightweight structures, stiffening structures, etc., at least in portions. In particular, it is expedient if the orthosis parts can be formed, in particular in one piece, with stiffening structures. Corresponding stiffening structures can, for example, be formed by rib structures or can comprise such structures.

In principle, at least two of the orthosis parts can be movable relative to each other in at least one translatory degree of freedom of movement and/or in at least one rotatory degree of freedom of movement—this can in particular be understood as a pivoting degree of freedom of movement about a horizontal pivot axis. In particular, it is possible that the third orthosis part can be fastened or is fastened to the second orthosis part so as to be movable relative to the second orthosis part. This can be particularly useful to enable a wearer to “get into” the orthosis more easily. The third orthosis part can thus be moved, for example, into a first orientation and/or position, i.e. in particular into a first pivot position, which allows a wearer to gain access to the second orthosis part, and into a second orientation and/or position, i.e. in particular into a second pivot position, which allows a wearer who has gotten into the first orthosis part to fasten the third orthosis part to the lower leg. In the second orientation and/or position, the third orthosis part typically extends or runs in a distal or proximal direction with respect to the leg of the wearer who has gotten into the second orthosis part, between the first orthosis part and the second orthosis part. The fastening of the orthosis parts to each other does not therefore fundamentally exclude a relative movement of at least two orthosis parts.

Corresponding degrees of freedom of movement can be realized by interacting joint or hinge structures, for example on the second and/or third orthosis part. Corresponding joint or hinge structures can also be formed in one piece or integrally, for example with the second orthosis part and/or third orthosis part, as part of an additive manufacturing process.

A further aspect of the invention relates to an orthosis part for an orthosis according to the first aspect of the invention. A corresponding orthosis part, which may be formed, for example, as a first, second or third orthosis part, is characterized in that it has, at least in portions, possibly completely, a geometric-constructive design which is individually configured on the basis of data describing a foot and/or a lower leg of a wearer. All explanations in conjunction with the orthosis according to the first aspect apply analogously to the orthosis part. Accordingly, the orthosis part is expediently manufactured additively at least in portions, possibly completely.

Another aspect of the invention relates to a method for manufacturing at least one orthosis part for an orthosis for a lower extremity of a wearer, in particular for a foot and a lower leg of a wearer, preferably for wearers with diabetic foot syndrome, i.e. in particular for an orthosis according to the first aspect of the invention.

The method basically comprises the following steps:
generating or providing wearer data describing a foot and/or a lower leg of a wearer of the orthosis to be produced, and
manufacturing at least one orthosis part based on the generated or provided wearer data.

All explanations in conjunction with the orthosis according to the first aspect apply analogously to the method.

The wearer data generated or provided in the first step may be acquisition data that can be acquired or are acquired via a suitable acquisition device. The corresponding acquisition device may be, for example, an optical acquisition device, such as an image acquisition device, i.e., for example, a camera or scanning device. Corresponding acquisition data may therefore be, for example, one-dimensional or multi-dimensional image data, i.e. in particular camera, scan, CT or MRI data, of a foot and/or a lower leg of a wearer. The acquisition data can, for example, be generated or provided by a doctor, technician, in particular an orthopedic technician, or possibly even by the wearer themself.

The wearer or acquisition data generated or provided in the first step may be stored in a local or global data storage facility, such as a network memory (cloud memory). A corresponding network memory may be accessible via, for example, a network-based access portal via a (mobile) terminal, such as a laptop, smartphone, tablet, etc., of a user. The wearer or acquisition data can be stored in a corresponding data storage facility, in particular classified according to specific features or feature categories, which relate to features or feature categories of the lower leg and/or foot and/or wearer.

It is conceivable that, between the step of generating or providing corresponding wearer data and the step of manufacturing at least one orthosis part on the basis of the generated or provided wearer data, the method comprises a step of processing the wearer data to generate construction data relating to the geometric-constructive design of the orthosis part to be produced. In this case, at least one orthosis part is manufactured on the basis of corresponding construction data. Corresponding construction data can, for example, be CAD data and/or slice data, or corresponding construction data can contain CAD data and/or slice data.

In all embodiments, the orthosis part can be manufactured using an additive manufacturing process. Thus, an additive manufacturing device implementing an additive manufacturing process can be used to manufacture the orthosis part. This can be, for example, a multi-jet fusion device implementing a multi-jet fusion process.

In all embodiments, there can be arranged or formed on the second orthosis part one or more fastening interfaces, via which a single-part or multi-part sole part forming a walking or standing surface of the second orthosis part or of the orthosis—a corresponding sole part can optionally also be designated or considered as an outsole part—can be fastened to the second orthosis part, in particular in a detachable manner and thus in a manner that can be replaced as required. Corresponding fastening interfaces can be manufactured additively at least in portions, possibly completely. Corresponding fastening interfaces can therefore be formed in particular in one piece with the second orthosis part.

Consequently, a second orthosis part can be provided with a corresponding sole part if required, in that a corresponding sole part is fastened to the second orthosis part via the fastening interfaces arranged or formed on the second orthosis part. Of course, a corresponding sole part can be provided with, in particular corresponding, fastening interfaces which cooperate with the fastening interfaces of the second orthosis part to form a stable, but as mentioned, possibly detachable fastening of the sole part to the second orthosis part.

Corresponding fastening interfaces of the second orthosis part can, for example, be arranged or formed in the area of a bottom side or underside of the second orthosis part. The number and arrangement of the fastening interfaces is basically freely selectable.

Regardless of their number and arrangement, corresponding fastening interfaces can be set up to enable positive and/or non-positive fastening of a corresponding sole part to the second orthosis part. The fastening of a corresponding sole part to the second orthosis part can thus be realized for example via form-fitting and/or frictionally engaging fastening types. For example, clamping and/or detent/snap-in and/or screw and/or plug-in and/or tensioning fastenings and thus clamping and/or detent/snap-in and/or screw and/or plug-in and/or tensioning elements realizing clamping and/or detent/snap-in and/or screw and/or plug-in and/or tensioning fastenings can be considered as fastening elements.

A corresponding sole part can be provided with specially designed surface portions. Corresponding surface portions, i.e. for example curved and/or sloping surface portions, can improve the rolling behavior of the sole part.

A corresponding sole part can be provided with a profiling, for example formed by a three-dimensional surface structuring, in particular in the area of its underside facing away from the second orthosis part when it is fastened to the second orthosis part as intended. A corresponding profiling can be configured with regard to a specific surface, in particular in order to offer a user special wearing properties on the particular surface, i.e. in particular a special standing and/or walking behavior. In this way, not only the wearing properties of the orthosis can be improved, but also safety-relevant aspects of the orthosis, for example by implementing an anti-slip device.

A corresponding sole part may be formed from or may comprise, at least in portions, possibly completely, an elastic material, such as a natural or synthetic elastomer material.

Lastly, it should be noted that a corresponding sole part can also be manufactured additively, at least in portions, possibly completely.

A corresponding sole part may also be designated or considered as a fourth orthosis part in all exemplary embodiments.

The method may further comprise the following steps:

providing a plurality of data describing a preconfigured geometric-constructive
design of an orthosis part—these may also be provided as standard data—in a local or global data storage facility,
selecting data describing a preconfigured geometric-constructive design of a particular orthosis part on the basis of the provided wearer data from the data storage facility,
modifying the selected data on the basis of the provided wearer data for the purpose of generating modified data,
fabricating the orthosis part on the basis of the modified data.

The method can therefore provide that data describing a preconfigured geometric-constructive design of a specific orthosis part are selected from a data storage facility on the basis of wearer data. The selection can be made on the basis of a comparison of corresponding wearer data with specific data stored in the data storage facility. A corresponding comparison may provide for a comparison of specific features described in the wearer data, i.e., for example, features of a lower leg and/or a foot, with, in particular, corresponding features described in the data stored in the data storage facility. A corresponding comparison can be carried out by a hardware- and/or software-implemented comparison device, which can have suitable comparison algorithms.

Once data describing a preconfigured geometric design of a particular orthotic component have been selected from the data storage facility, the data can be modified based on the provided wearer data to produce modified data. Modification may include, for example, adjusting specific parameters, such as geometric-constructive parameters, of the preconfigured data with respect to corresponding parameters of the wearer data. Such modification may be performed by a suitable hardware- and/or software-implemented data modification device, which may have suitable data modification algorithms.

The modification of the data may thus result in an adaptation of the data on the basis of the provided wearer data in order to generate construction data relating to the geometric-constructive design of the orthosis to be produced. The method may therefore comprise a step of adapting the data describing the preconfigured geometric-constructive design of an orthosis part on the basis of the provided wearer data in order to generate construction data relating to the geometric-constructive design of the orthosis part to be produced. The modified data can form the basis for corresponding construction data or can be processed into such.

In principle, it is therefore possible to provide data describing a preconfigured geometric-constructive design of an orthosis part, the data being adapted on the basis of the provided wearer data in order to generate construction data relating to the geometric-constructive design of the orthosis part to be manufactured. This procedure can, if necessary, considerably simplify the computational effort for generating corresponding construction data.

Corresponding construction data can describe the orthosis part to be manufactured in all embodiments with a negative offset (undersize), in particular with regard to a functional element to be fastened to the orthosis part, such as a buffer and/or support element, which can specifically be, for example, an elastomer element, a foam element, etc. In this way, the circumstance can be taken into account that an orthosis part can be further provided with at least one functional element, such as a corresponding buffer and/or support element. In this case, a corresponding orthosis part can be understood as a semi-finished product which is to be subjected to one or more further process steps for the production of an orthosis.

A corresponding buffer and/or support element can also be provided, at least in portions, with at least one functional layer, such as a textile layer, in particular a breathable textile layer. In all embodiments, a buffer and/or support element can enable better interaction between the orthosis and the wearer.

The invention is explained in greater detail by means of exemplary embodiments in the drawings, in which:

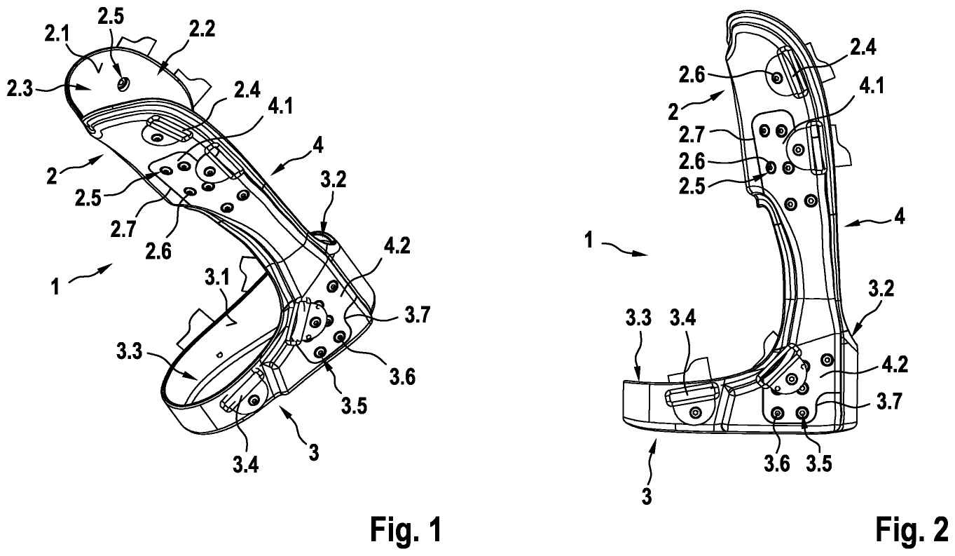

FIGS. 1-3 each show a schematic illustration of an orthosis according to a first exemplary embodiment;

FIGS. 4-7 each show a schematic illustration of an orthosis according to a further exemplary embodiment;

FIG. 8 shows a schematic illustration of an orthosis according to a further exemplary embodiment;

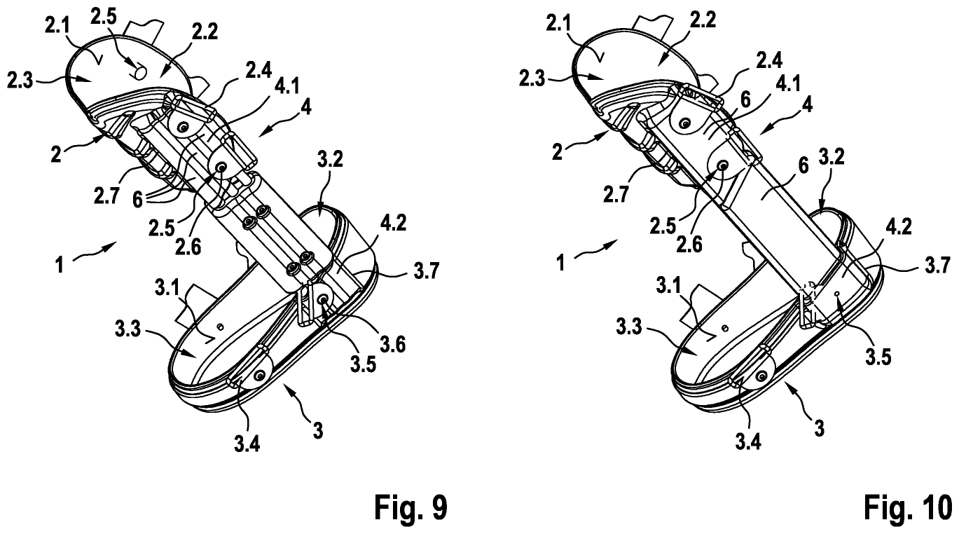

FIG. 9 shows a schematic illustration of an orthosis according to a further exemplary embodiment;

FIG. 10 shows a schematic illustration of an orthosis according to a further exemplary embodiment;

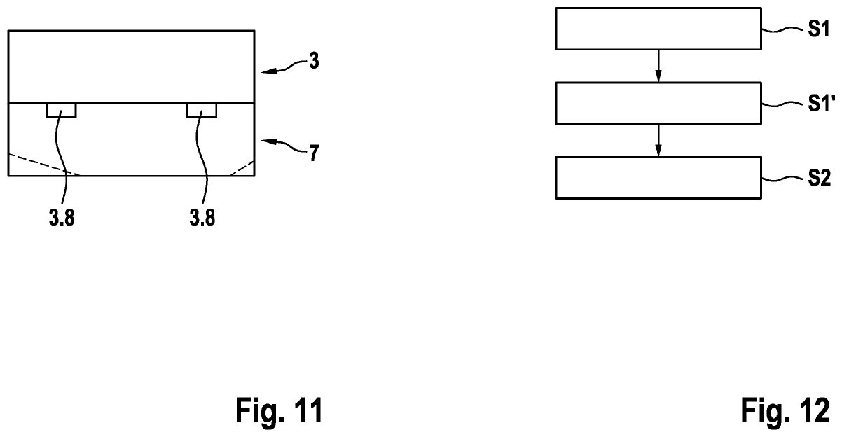

FIG. 11 shows a schematic illustration of a second orthosis part according to a further exemplary embodiment;

FIG. 12 shows a block diagram illustrating a method for producing an orthosis part according to an exemplary embodiment;

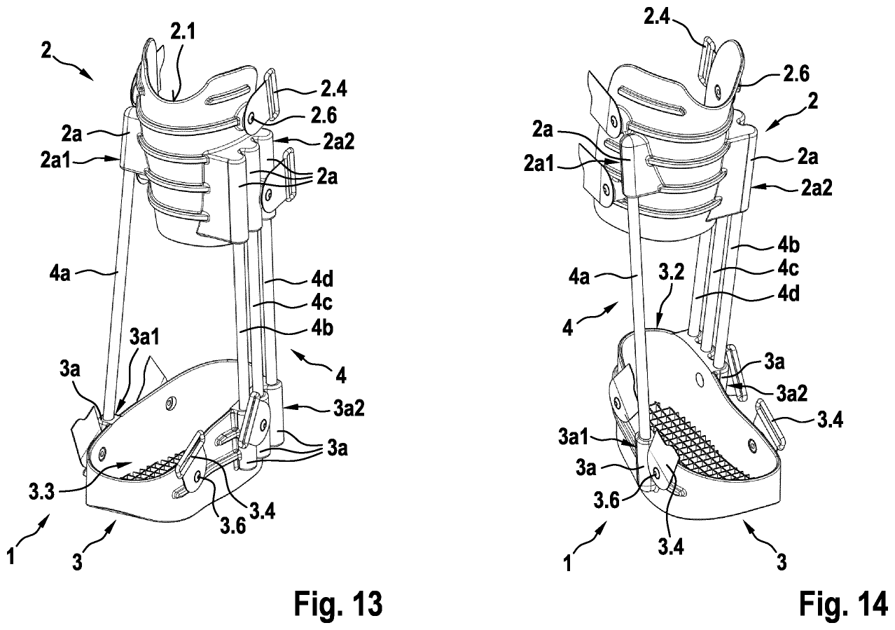

FIGS. 13 and 14 each show a schematic illustration of an orthosis according to a further exemplary embodiment;

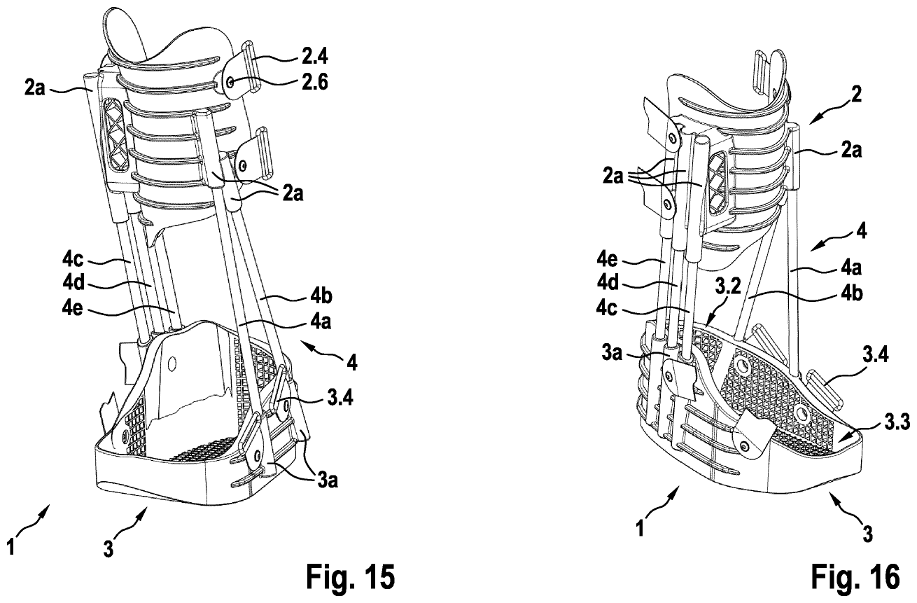

FIGS. 15 and 16 each show a schematic illustration of an orthosis according to a further exemplary embodiment; and

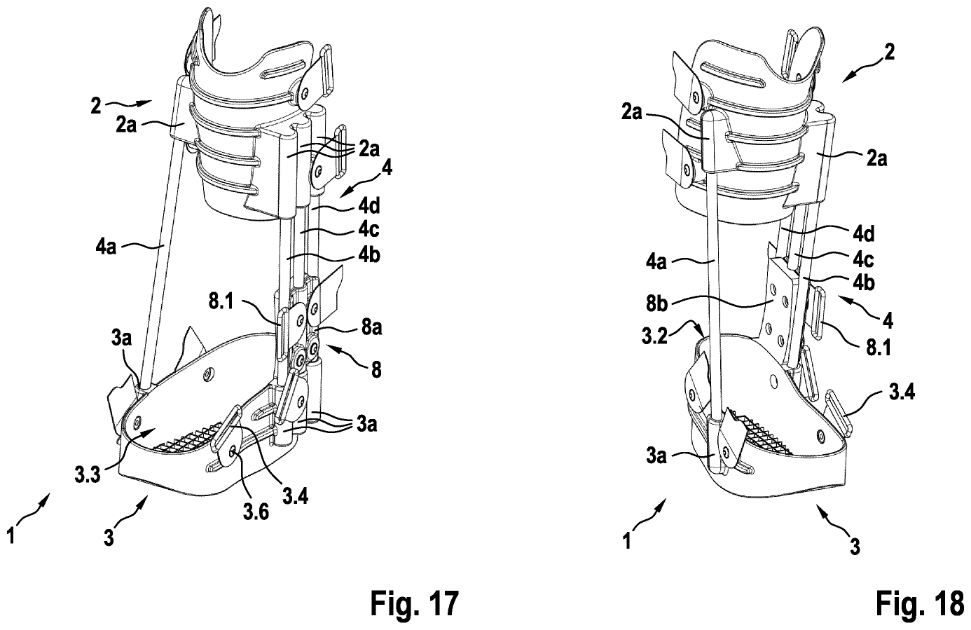

FIG. 17, 18 each show a schematic illustration of an orthosis according to a further exemplary embodiment.

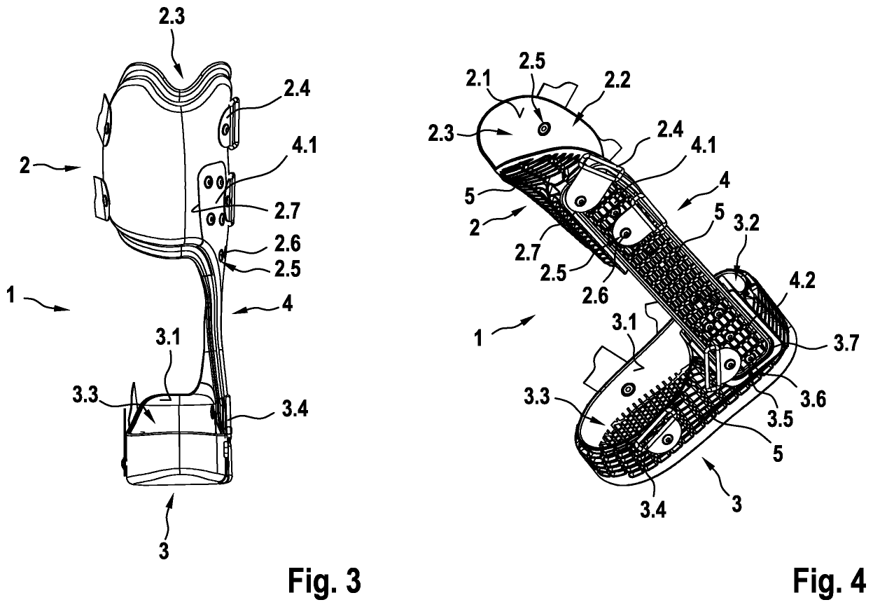

FIG. 1 shows a schematic illustration of an orthosis 1 according to an exemplary embodiment in a perspective view. FIG. 2 and FIG. 3, which are to be assigned to the same exemplary embodiment, show the orthosis 1 in a side view (FIG. 2) and in a front view (FIG. 3).

The orthosis 1 is an orthosis for a lower extremity of a wearer, in particular for a foot and a lower leg of a wearer, preferably for wearers with diabetic foot syndrome. The orthosis 1 generally represents a component or group of components belonging to the field of technical orthopedics and is particularly configured for the treatment of diabetic foot syndrome or a neuropathic osteoarthropathy relating to the foot. The orthosis 1 is thus intended in particular for wearers or patients with diabetic foot syndrome or a neuropathic osteoarthropathy relating to the foot.

On the basis of FIGS. 1-10, i.e. in particular FIGS. 1-18, it can be seen that the orthosis 1 comprises three orthosis parts 2-4, i.e. a first orthosis part 2, a second orthosis part 3 and a third orthosis part 4. The orthosis 1 is thus constructed in a multi-part or modular manner:

The orthosis 1 comprises the first orthosis part 2, which can be arranged or which is to be arranged on a lower leg, i.e. in particular on or in the region of a shin, of a wearer. With regard to its functional as well as constructive design, the first orthosis part 2 is thus configured in such a way that it can be arranged on a lower leg of a wearer or is arranged on a lower leg of the wearer when the orthosis 1 is worn as intended. The first orthosis part 2 thus extends at least in portions along and/or around a lower leg of a wearer when the orthosis 1 is worn as intended. The first orthosis part 2 can enclose the lower leg of the wearer at least in portions, possibly completely. The first orthosis part 2 thus has one or more contact regions 2.1 or areas which, when the orthosis 1 is worn as intended, lie against the lower leg of a wearer. The dimensions of the first orthosis part 2 are typically adapted to the dimensions, i.e. in particular width, length, circumference, etc., of the lower leg. The first orthosis part 2 can also be designated or considered as a lower leg part.

The first orthosis part 2 comprises a main body, not specified in greater detail, which has a receiving area 2.3, in particular accessible via a dorsally oriented access area 2.2, for at least partially receiving a lower leg or a lower leg portion of a wearer. The receiving area 2.3 can be provided with corresponding contact areas 2.1 or surfaces which, when the orthosis is worn as intended, lie against or at least partially enclose the lower leg of a wearer. The access area 2.2 is selected in such a way that it is as easy as possible to put on and take off the first orthosis part 2, i.e. in particular is as easy as possible to get in and out of the first orthosis part 2.

The main body of the first orthosis part 2 has a geometric-constructive design that is individually configured on the basis of data describing a lower leg of a wearer. Specifically, the main body can, for example, have a geometric-constructive design that surrounds a lower leg of a wearer, in particular on the shin side below the patella, at least in portions. The main body can thus have a semi-shell-like or -shaped basic form individually adapted to a lower leg of a wearer. Viewed cross-sectionally, the main body can have a U- or V-shaped basic shape or geometry individually adapted to a lower leg of a wearer. The main body, i.e. in particular the various contact regions or areas of the main body, can surround the lower leg of a wearer at least in portions, in particular in a form-fitting manner. In this way, an undesired rotation of the wearer's lower leg can be prevented or made more difficult with the orthosis 1.

One or more, in particular tab-like or -shaped or eyelet-like or -shaped, interfaces 2.4 can be arranged or formed on the main body for fastening a, in particular band-like or -shaped, fixing means (not shown) for fixing, in particular as required, a lower leg portion of a wearer in the receiving area 2.3. A corresponding interface 2.4 can be formed in one piece (integrally) with the main body so that it forms an integral part of the main body.

Furthermore, a plurality of, in particular hole-like or -shaped and/or thread-like or -shaped interfaces 2.5 are arranged or formed on the main body of the first orthosis part 2 for fastening a fastening means 2.6, in particular acting in a form-locking and/or frictionally engaging manner, for fastening, in particular releasably, the third orthosis part 4 to the first orthosis part 2. A corresponding interface 2.5 can be formed in one piece (integrally) with the main body so that it forms an integral part of the main body.

As shown in FIGS. 1-10, a plurality of corresponding interfaces 2.5 are arranged or formed in a defined arrangement on the first orthosis part 2. The arrangement of the interfaces 2.5 is selected in such a way that it allows a desired arrangement and orientation of the third orthosis part 4 on or relative to the first orthosis part 2.

The orthosis 1 further comprises the second orthosis part 3 which can be arranged or which is to be arranged on a foot of a wearer. With regard to its functional as well as constructive design, the second orthosis part 3 is thus configured in such a way that it can be arranged on a foot of a wearer or is arranged on a foot of the wearer in the intended worn state of the orthosis 1. The second orthosis part 3 thus extends at least in portions along and/or around a foot of a wearer when the orthosis 1 is worn as intended. The second orthosis part 3 can enclose the foot of a wearer at least in portions, if necessary, completely. The second orthosis part 3 thus has one or more contact regions 3.1 or areas which, when the orthosis 1 is worn as intended, lie against the foot of a wearer. The dimensions of the second orthosis part 3 can be adapted to the dimensions of the foot, i.e. in particular width, length, circumference, etc. The second orthosis part 3 can also be designated or considered as a foot part.

The second orthosis part 3 comprises an unspecified main body which has a receiving area 3.3, accessible in particular via a dorsally oriented access area 3.2, for at least partially receiving a foot or a foot portion of a wearer. The actual entry into the orthosis 1 can, however, be made from the front. The receiving area 3.3 of the second orthosis part 3 can be provided with corresponding contact regions 3.1 or areas which, when the orthosis is worn as intended, lie against or at least partially enclose the foot of a wearer. The access area 3.2 is selected in such a way that it is as easy as possible to put on and take off the second orthosis part 3, i.e. in particular is as easy as possible to get in and out of the second orthosis part 3.

In addition to corresponding interfaces, which allow the orthosis parts 2-4 to be fastened on or to each other, positioning devices 2.7, 3.7 can also be formed on one or more orthosis parts and allow an exact positioning—through this, if necessary, pre-assembly positions of the orthosis parts 2-4 can be realized—of the orthosis parts 2-4 to be fastened to each other.

It can be seen from FIG. 1 that a positioning device 2.7 can be arranged or formed on the first orthosis part 2 and allows exact positioning of the third orthosis part 4 to be fastened on or to the first orthosis part 2. The positioning device 2.7 is formed by way of example by a recess or depression in the first orthosis part 2, at or in which the upper portion of the third orthosis part 4 forming the interface 4.1 can be inserted. As FIG. 1 shows, a corresponding recess or depression can be configured geometrically and structurally in such a way that it has a contour that is the opposite of the contour of the corresponding portion of the third orthosis part 4. A reverse configuration is also conceivable.

It can be seen from FIG. 1 that a positioning device 3.7 can be arranged or formed on the second orthosis part 3 and allows exact positioning of the third orthosis part 4 to be fastened on or to the second orthosis part 3. The positioning device 3.7 is formed by way of example by a recess or depression in the second orthosis part 3, at or in which the lower portion of the third orthosis part 4 forming the interface 4.2 can be inserted. As FIG. 1 shows, a corresponding recess or depression can be configured in geometric-constructive terms in such a way that it has a contouring that is the opposite of the contour of the corresponding portion of the third orthosis part 4. Again, a reverse configuration is also conceivable.

The main body of the second orthosis part 3 has a geometric-constructive design which is individually configured on the basis of data describing a foot of a wearer; the geometric-constructive design of the main body of the second orthosis part 3 is therefore. Specifically, the main body can, for example, have a geometric-constructive design which surrounds a foot of a wearer, in particular on the sole side of the foot and/or on the (outer and/or inner) instep side and/or on the dorsum side, at least in portions. The main body can therefore have, for example, a shoe-like basic shape or geometry individually adapted to a foot of a wearer. In particular, the main body can have a basic shape or geometry of an orthopedic shoe, in particular a shoe for “diabetic feet”, which is individually adapted to a foot of a wearer.

One or more, in particular tab-like or -shaped or eyelet-like or -shaped, interfaces 3.4 can be arranged or formed on the main body for fastening a, in particular band-like or -shaped, fixing means for fixing, in particular as required, a foot portion of a wearer in the receiving area 3.2. A corresponding interface 3.4 can be formed in one piece (integrally) with the main body so that it forms an integral part of the main body.

Furthermore, a plurality of, in particular hole-like or -shaped and/or thread-like or -shaped interfaces 3.5 are arranged or formed on the main body for fastening a fastening means 3.6, in particular acting in a form-locking and/or frictionally engaging manner, for fastening, in particular releasably, the third orthosis part 4 to the second orthosis part 3. A corresponding interface 3.5 can be formed in one piece (integrally) with the main body so that it forms an integral part of the main body. As shown in the Fig., a plurality of corresponding interfaces 3.5 are arranged or formed in a defined arrangement on the second orthosis part 3. The arrangement of the interfaces 3.5 is selected in such a way that it allows a desired arrangement and orientation of the third orthosis part 4 on or relative to the second orthosis part 3.

The orthosis 1 further comprises the third orthosis part 4 connecting the first orthosis part 2 and the second orthosis part 3 to one another. The third orthosis part 4 is thus configured with regard to its functional as well as constructive design in such a way that it can be arranged at least in portions between the first and the second orthosis parts 2, 3 in order to connect the first orthosis part 2 to the second orthosis part 3. The third orthosis part 4 thus comprises at least one first interface 4.1, via which it can be fastened to the first orthosis part 2, i.e. in particular to a corresponding interface provided for this purpose, and at least one second interface 4.2, via which it can be fastened to the second orthosis part 3, i.e. in particular to a corresponding interface provided for this purpose. The third orthosis part 4 extends, as FIGS. 1-10 indicate, in the intended worn state of the orthosis 1 in a distal or proximal direction with respect to the leg of the wearer between the first orthosis part 2 and the second orthosis part 3. The third orthosis part 4 can be arranged medially or laterally or possibly dorsally on the leg of a wearer in the intended worn state of the orthosis 1. The third orthosis part 4 can also be designated or considered as a connecting part.

The third orthosis part 4 has an elongate, in particular strut-like or -shaped, geometrically-constructive main body which is not specified in greater detail. In the intended worn state of the orthosis 1, the main body extends between the first and second orthosis parts 2, 3. This results in a monolateral or monomedial arrangement of the third orthosis part 4, as the main body only extends along one side of the lower leg of the wearer in the intended worn state of the orthosis 1.

The third orthosis part 4 may be configured to fulfil all strength-related aspects, so that it may be sufficient to subject only this component to a test for sufficient strength. The first and second orthosis parts 2, 3 can be configured to fit the third orthosis part 4 and to be patient-specific and additively manufactured.

If the main body is not provided with desired structural properties, i.e. in particular a desired stiffness, due to its geometric-constructive design, the third orthosis part 4 can comprise one or more stiffening elements 6 extending in longitudinal direction (cf. for example FIGS. 9 and 10). Corresponding stiffening elements 6 can be formed from a mechanically stiff material, such as metal, composite material, in particular glass or carbon fiber material, or a corresponding material structure, or can comprise such a material structure.

The orthosis parts 2-4 are thus set up to cooperate with each other to form the orthosis 1 and in particular can be fastened or are fastened to each other for this purpose. In particular, the first orthosis part 2 can be fastened to the second orthosis part 3 via the third orthosis part 4, and vice versa.

The fastening of the third orthosis part 4 to the first orthosis part 2 and/or to the second orthosis part 3 can be realized, for example, by means of form-fitting and/or frictionally engaging fastening types. For example, clamping and/or detent/snap-in and/or screw and/or plug-in and/or tensioning fastenings and thus corresponding clamping and/or detent/snap-in and/or screw and/or plug-in and/or tensioning fastenings realizing clamping and/or detent/snap-in and/or screw and/or plug-in and/or tensioning elements can be considered as fastening elements.

In all exemplary embodiments, the fastening of the third orthosis part 4 to the first orthosis part 2 and/or to the second orthosis part 3 can be detachable (without damage or destruction). The fastening of the third orthosis part 4 to the first orthosis part 2 and/or to the second orthosis part 3 can therefore be cancelled and restored if necessary.

In the first exemplary embodiment, the first orthosis part 2 and the second orthosis part 3 have an individually configured geometric-constructive design based on data describing a foot and/or a lower leg of a wearer. The first and the second orthosis part thus have an individually configured geometric-constructive design and thus a high degree of individualization. The first and the second orthosis part 2, 3 are therefore each an individually configurable or individually configured individual part for a specific limb, i.e. in particular a specific foot or a specific lower leg, of a specific wearer. The orthosis parts 2, 3 can represent an individually configured (negative) model of a lower leg or foot portion of a specific wearer, on which they are to be arranged. This allows an individually configured and thus exact fit of the orthosis parts 2, 3 that is specific to the wearer or the wearer's body part.

The individually configured geometric-constructive design of the orthosis parts 2, 3 was generated, as mentioned, on the basis of data describing a foot and/or a lower leg of a wearer at least in portions, possibly completely. Corresponding data can be, for example, acquisition data that can be acquired or are acquired via a suitable acquisition device. A corresponding acquisition device can be or can comprise, for example, an optical acquisition device, such as an image acquisition device, i.e., for example, a camera or scanning device. Corresponding data can therefore be, for example, one-dimensional or multi-dimensional image data, i.e. in particular camera, scan, CT or MRI data of a foot and/or lower leg of a wearer.

The individually configured geometric-constructive design of the orthosis parts 2, 3 can be realized expediently in terms of production technology, in particular with additive manufacturing processes—sometimes also referred to as “3D printing”. Thus, the orthosis parts 2, 3 are manufactured additively, i.e. in an additive manufacturing process. Additive manufacturing allows components with special structural, i.e. both functional and constructive, properties to be formed, which with conventional manufacturing processes cannot be realized or can only be realized to a limited extent.

In principle, all additive manufacturing processes can be considered. Only by way of example is reference made to the multi-jet fusion process in conjunction with the exemplary embodiment shown in FIGS. 1-10. The orthosis parts 2, 3 can be made of a plastic, such as polyamide, for example.

In principle, however, it is true for all orthosis parts 2-4 that these can be formed with or can include functional structures, such as lightweight structures, stiffening structures, etc., at least in portions.

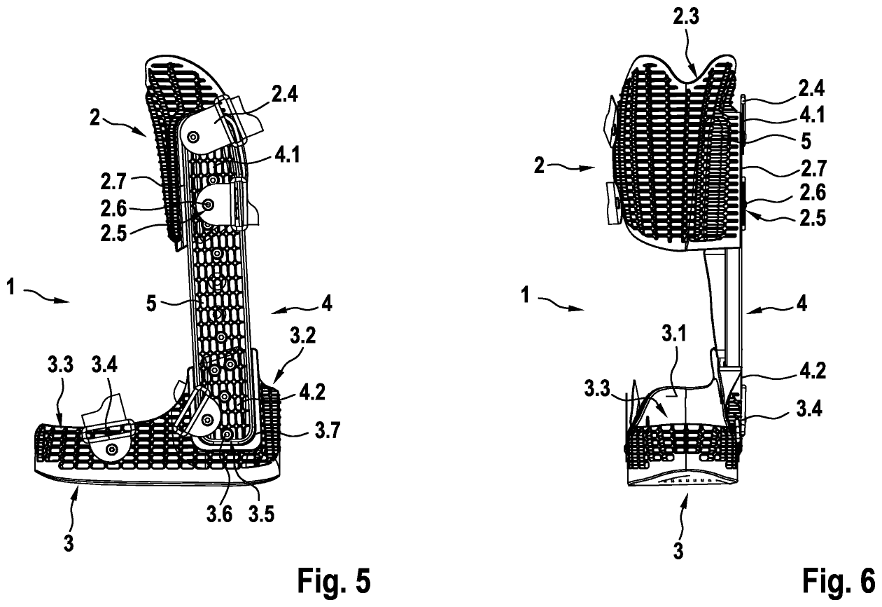

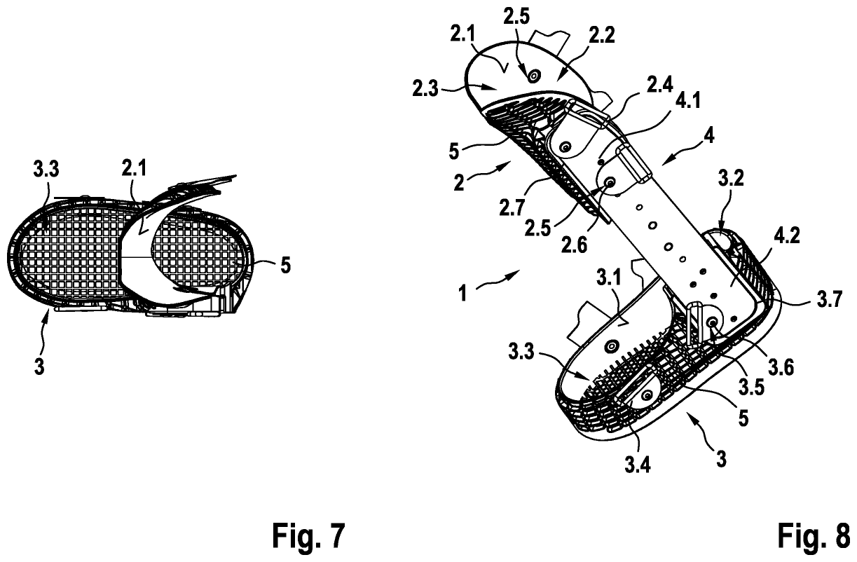

FIG. 4 shows a schematic illustration of an orthosis 1 according to a further exemplary embodiment in a perspective view. FIGS. 5-7, which are to be assigned to the same exemplary embodiment, show the orthosis 1 in a side view (FIG. 5), in a front view (FIG. 6) and in a plan view (FIG. 7).

The exemplary embodiment shown in FIGS. 4-7 differs from the exemplary embodiment shown in FIGS. 1-3 in that all orthosis parts 2-4 are additively manufactured. Thus, in this exemplary embodiment, the third orthosis part 4 is also additively manufactured.

It can be seen from FIGS. 4-7 that the orthosis parts 2-4 can also optionally be formed with rib structures 5 serving as stiffening structures, which give them the required structural properties. Contrary to FIGS. 1-10, corresponding stiffening structures can also be formed only on individual orthosis parts 2-4.

FIG. 8 shows a schematic illustration of an orthosis 1 according to a further exemplary embodiment in a perspective view.

The exemplary embodiment shown in FIG. 8 differs from the exemplary embodiment shown in FIGS. 4-7 in that here, instead of an additively manufactured third orthosis part 4, a third orthosis part 4 with a rail-like or -shaped main body made of a rigid material, such as metal or a carbon fiber composite material, is provided. The main body extends distally or proximally.

FIG. 9 shows a schematic illustration of an orthosis 1 according to a further exemplary embodiment in a perspective view.

The exemplary embodiment shown in FIG. 9 differs from the exemplary embodiments shown in the previous Fig. in that a third orthosis part 4 is provided here with a main body comprising a plurality of strut-like or -shaped stiffening elements 6 made of a rigid material, such as metal or a carbon fiber composite material. The stiffening elements 6 are arranged in parallel and extend distally and proximally following the main body.

FIG. 10 shows a schematic illustration of an orthosis 1 according to a further exemplary embodiment in a perspective view.

The exemplary embodiment shown in FIG. 10 differs from the exemplary embodiments shown in the previous Fig. in that a third orthosis part 4 is provided here with a main body comprising a plurality of rectangular stiffening elements 6 made of a rigid material, such as metal or a carbon fiber composite material, which may be inserted one inside the other. The stiffening elements 6 are arranged in series and extend distally or proximally following the main body.

In principle, it is true for all exemplary embodiments that at least two of the orthosis parts 2-4 can be movable relative to each other in at least one degree of freedom of movement, which can be understood in particular to be a degree of freedom of pivoting movement about a horizontal pivot axis. In particular, it is possible that the third orthosis part 4 can be fastened or is fastened to the second orthosis part 2 in a movable manner relative to the second orthosis part 2. This can be particularly expedient in order to allow a wearer to “get into” the orthosis 1 more easily. The third orthosis part 4 can thus be moved into a first orientation and/or position, i.e. in particular into a first pivot position, which allows a wearer to gain access to the first orthosis part 3, and into a second orientation and/or position, i.e. in particular into a second pivot position, which allows a wearer who has gotten into the second orthosis part 3 to fasten the third orthosis part 4 to the lower leg. In the second orientation and/or position, the third orthosis part 4 typically extends or runs in a distal or proximal direction with respect to the wearer's leg between the first orthosis part and the second orthosis part 3.

Corresponding degrees of freedom of movement can be realized by interacting joint or hinge structures on the second orthosis part 3 and/or third orthosis part 4. Corresponding joint or hinge structures can also be formed in one piece or integrally with the second orthosis part 3 and/or the third orthosis part 4 as part of an additive manufacturing process.

The orthosis parts 2-4 shown in FIGS. 1-10, i.e. in particular the first and second orthosis parts 2, 3, can be produced by the method described below for manufacturing at least one orthosis part for an orthosis 1 for a lower extremity of a wearer, in particular for a foot and a lower leg of a wearer, preferably for wearers with a diabetic foot syndrome or a neuropathic osteoarthropathy relating to the foot.

FIG. 11 shows a further exemplary embodiment of a second orthosis part 3. In the exemplary embodiment shown in FIG. 11, there are arranged or formed on the second orthosis part 3 fastening interfaces 3.8, which are indicated purely schematically and via which a single-part or multi-part sole part 7 forming a walking or standing surface of the second orthosis part 3 or of the orthosis 1 can be fastened to the second orthosis part 3, in particular in a detachable and thus replaceable manner if required.

Consequently, the second orthosis part 3 can be provided with a corresponding sole part 7, if required, in that a corresponding sole part 7 is fastened to the second orthosis part 3 via the fastening interfaces 3.8 arranged or formed on the second orthosis part 3. Of course, a corresponding sole part 7 can be provided with, in particular corresponding, fastening interfaces (not shown), which are set up to cooperate with the fastening interfaces 3.8 of the second orthosis part 3 to form a stable, but, as mentioned, possibly detachable, fastening of the sole part 7 to the second orthosis part 3.

It can be seen from FIG. 11 that corresponding fastening interfaces 3.8 can be arranged or formed, for example, in the region of an underside of the second orthosis part 3. The number and arrangement of the fastening interfaces 3.8 is freely selectable in principle.

Irrespective of their number and arrangement, corresponding fastening interfaces 3.8 can be set up to allow positive and/or non-positive fastening of a corresponding sole part 7 to the second orthosis part 3. The fastening of a corresponding sole part 7 to the second orthosis part 3 can thus be realized, for example, by means of form-fitting and/or frictionally engaging fastening types. For example, clamping and/or detent/snap-in and/or screw and/or plug-in and/or tensioning fastenings can be used as fastening types, and thus clamping and/or detent/snap-in and/or screw and/or plug-in and/or tensioning fastenings can be used as fastening elements 3.8.

As schematically indicated by the dotted lines, a corresponding sole part 7 can be provided with specially configured surface portions, in particular with regard to a special standing or rolling behavior—shown by way of example as sloping surfaces in FIG. 11. Corresponding surface portions can improve the rolling and/or standing behavior of the sole part 7 and thus of the orthosis 1.

A corresponding sole part 7 can be provided with a profiling, for example formed by a three-dimensional surface structuring, in particular in the region of its underside facing away from the second orthosis part 3 when it is fastened to the second orthosis part 3 as intended. A corresponding profiling can be configured with regard to a specific surface, in particular in order to offer a user special wearing properties on the respective surface, i.e. in particular a special standing and/or rolling behavior. In this way, not only can the wearing properties of the orthosis 1 be improved, but also safety-relevant aspects of the orthosis 1, for example by implementing an anti-slip device.

A corresponding sole part 7 may be formed from or may comprise, at least in portions, possibly completely, an elastic material, such as a natural or synthetic elastomer material.

Lastly, it should be noted that a corresponding sole part 7 can also be manufactured additively at least in portions, possibly completely.

In all exemplary embodiments, a corresponding sole part 7 can also be designated or considered as a fourth orthosis part.

The method schematically illustrated in the block diagram shown in FIG. 12 basically comprises the following steps:
generating or providing wearer data describing a foot and/or a lower leg of a wearer of the orthosis 1 to be produced (step S1), and
manufacturing at least one orthosis part 2-4 on the basis of the generated or provided wearer data (step S2).

The wearer data generated or provided in the first step S1 may be acquisition data that can be acquired or are acquired via a suitable acquisition device. The corresponding acquisition device may be, for example, an optical acquisition device, such as an image acquisition device, i.e., for example, a camera or scanning device. Corresponding acquisition data may therefore be, for example, one-dimensional or multi-dimensional image data, i.e. in particular camera or scan data, of a foot and/or a lower leg of a wearer. The acquisition data can, for example, be generated or provided by a doctor, technician, in particular an orthopedic technician, or possibly even by the wearer themself.

The wearer or acquisition data generated or provided in the first step S1 may be stored in a local or global data storage facility, such as a network memory (cloud memory). A corresponding network memory may be accessible via, for example, a network-based access portal via a (mobile) terminal, such as a laptop, smartphone, tablet, etc., of a user. The wearer or acquisition data can be stored in a corresponding data storage facility, in particular classified according to specific features or feature categories, which relate to features or feature categories of the lower leg and/or foot and/or wearer.

It is conceivable that between the step S1 of generating or providing corresponding wearer data and the step S2 of manufacturing at least one orthosis part on the basis of the generated or provided wearer data, the method comprises a step S1′ of processing the wearer data to generate construction data relating to the geometric-constructive design of the orthosis part 2-4 to be produced. In this case, at least one orthosis part 2-4 is manufactured on the basis of corresponding construction data. Corresponding construction data can, for example, be CAD data and/or slice data, or corresponding construction data can contain CAD data and/or slice data.

In all exemplary embodiments, the particular orthosis part 2-4 can be manufactured using an additive manufacturing process. An additive manufacturing device implementing an additive manufacturing process can therefore be used to manufacture an orthosis part 2-4. This can be, for example, a multi-jet fusion device implementing a multi-jet fusion process.

The method may further comprise the following steps:
providing a plurality of data describing a preconfigured geometrical-constructive design of an orthosis part—these may optionally also be referred to as standard data—in a local or global data storage facility,
selecting data describing a preconfigured geometrical-constructive design of a particular orthosis part 2-4 on the basis of the provided wearer data from the data storage facility,
modifying the selected data based on the provided wearer data to generate modified data,
manufacturing the orthosis part 2-4 based on the modified data.

The method can thus provide that, on the basis of wearer data, data describing a preconfigured geometric-constructive design of a specific orthosis part 2-4 are selected or chosen from a data storage facility. The selection can be made on the basis of a comparison of corresponding wearer data with specific data stored in the data storage facility. A corresponding comparison may provide for a comparison of specific features described in the wearer data, i.e., for example, features of a lower leg and/or a foot, with, in particular, corresponding features described in the data stored in the data storage facility. A corresponding comparison can be carried out by a hardware- and/or software-implemented comparison device, which can have suitable comparison algorithms.

After selecting data describing a pre-configured geometric-constructive design of a particular orthosis part 2-4, these data can be modified based on the provided wearer data to generate modified data. Modification may include, for example, adjusting specific parameters, such as geometric-constructive parameters, of the pre-configured data with respect to corresponding parameters of the wearer data. A corresponding modification may be performed by a suitable hardware- and/or software-implemented data modification device, which may have suitable data modification algorithms.

The modification of the data can therefore result in an adaptation of the data on the basis of the provided wearer data in order to generate construction data relating to the geometric-constructive design of the orthosis part 2-4 to be produced. The method can therefore comprise a step of adapting the data describing the preconfigured geometric-constructive design of an orthosis part 2-4 on the basis of the provided wearer data in order to generate construction data relating to the geometric-constructive design of the orthosis part 2-4 to be produced. The modified data can form the basis for corresponding construction data or can be processed into such.

In principle, it is therefore possible that data describing a preconfigured geometric-constructive design of an orthosis part 2-4 are provided and are adapted, on the basis of the provided wearer data, in order to generate construction data relating to the geometric-constructive design of the orthosis part 2-4 to be produced.

Corresponding construction data can describe the orthosis part 2-4 to be produced in all embodiments with a negative offset (undersize), in particular with regard to a functional element to be fastened to the orthosis part 2-4, such as a buffer and/or support element, which can specifically be, for example, an elastomer element or a foam element. In this way, it can be taken into account that an orthosis part 2-4 can be further provided with at least one functional element, such as a corresponding buffer and/or support element. In this case, an orthosis part 2-4 can be understood as a semi-finished product which is to be subjected to one or more further process steps for production of an orthosis 1.

A corresponding buffer and/or support element can also be provided, at least in portions, with at least one functional layer, such as a textile layer, in particular a breathable textile layer. In all embodiments, a buffer and/or support element can allow better interaction between the orthosis and the wearer.

In conjunction with the exemplary embodiments according to FIGS. 13-18, it can be seen that the third orthosis part 4 can be formed by one or more strut elements 4a-4x or can comprise one or more strut elements 4a-4x.

FIGS. 13-18 show that the first orthosis part 2 and the second orthosis part 3 can each comprise at least one receiving or fastening area 2a, 3a for receiving or fastening at least one strut element 4a-4x, i.e. in particular for receiving or fastening a receiving or fastening portion 4a1-4x1 of a corresponding strut element 4a-4x, which cooperates with a corresponding receiving or fastening area 2a, 3a, on the particular orthosis part 2, 3. Corresponding receiving or fastening areas 2a, 3a can be formed by plug-in receptacles, as shown in the Fig. by way of example. Alternatively or additionally, clamping, tensioning or detent receptacles are conceivable. Alternatively or additionally, gluing or weld receptacles are conceivable; a corresponding strut element 4a-4x can thus be glued or welded to a corresponding receptacle or fastening area 2a, 3a, for example by means of a single or multi-component adhesive. Basically, the orthosis-part-side receiving or fastening areas 2a, 3a allow the strut elements 4a-4x to be detachably or non-detachably received or fastened on respective orthosis parts 2, 3.

The strut elements 4a-4x can also serve as stiffening elements due to their structural properties, i.e. in particular their stiffness. The structural properties, i.e. in particular the stiffness, of the strut elements 4a-4x can result from the material forming the strut elements 4a-4x or the material structure forming the strut elements 4a-4x. For example, the strut elements 4a-4x can be made of metal, fiber-reinforced plastic, fiber composite materials, i.e. in particular glass or carbon fiber composite materials.

Specific configurations of the orthosis 1 with at least one third orthosis part 4 configured in a strut-like or -shaped manner will be explained in greater detail in the following with reference to FIGS. 13-18 as examples. It is evident that the strut elements 4a-4x can be arranged or are arranged medially and/or laterally, i.e. generally to the side, on the leg of a wearer when the orthosis 1 is worn as intended.

The configuration of the orthosis 1 according to the exemplary embodiment shown in FIG. 13, 14 in two different perspective views provides that a first strut element 4a is arranged extending laterally (first side) between the first and the second orthosis parts 2, 3 when the orthosis 1 is worn as intended and a plurality of further strut elements 4b-4d are arranged extending medially (second side) between the first and the second orthosis parts 2, 3 when the orthosis 1 is worn as intended. Thus, at least one strut element 4a-4d is present both laterally and medially, so that strut elements 4a-4d are arranged or oriented opposite each other. The first strut element 4a is fastened with a first receiving or fastening portion (not designated) to or in a laterally oriented receiving or fastening area 2a1 of the first orthosis part 2 and with a second receiving or fastening portion (not designated) to or in a laterally oriented receiving or fastening area 3a1 of the second orthosis part 3. The further strut elements 4b-4d are fastened with respective first receiving or fastening portions to or in a medially oriented receiving or fastening area 2a2 of the first orthosis part 2 and with respective second receiving or fastening portions to or in a medially oriented receiving or fastening area 3a2 of the second orthosis part 3. The medially arranged strut elements 4b-4d are arranged in parallel by way of example.

The configuration shown in FIGS. 13 and 14 thus specifically provides that a plurality of strut elements 4b-4e are arranged extending medially between the first and the second orthosis parts 2, 3 when the orthosis 1 is worn as intended and a (single) strut element 4a is arranged extending laterally between the first and the second orthosis parts 2, 3 when the orthosis 1 is worn as intended. A reverse configuration would also be conceivable.

The configuration of the orthosis 1 according to the exemplary embodiment shown in FIGS. 15 and 16 in two different perspective views provides, in contrast to the exemplary embodiment according to FIGS. 13 and 14, that, again with reference to the condition of the orthosis 1 when worn as intended, a plurality of first strut elements 4a are also present laterally. The orthosis 1 thus has a plurality of first strut elements 4a. It is evident that these are not arranged parallel to the medially arranged strut elements 4b-4e, but at an angle. In this way, space is created, for example, for the arrangement of the interfaces on the second orthosis part 3.

For all configurations, it is basically true that medially and/or laterally arranged strut elements 4a-4x can be arranged or oriented in different spatial directions. By selecting different spatial directions in which each strut element 4a-4x extend, it is also possible to influence the structural properties of the orthosis 1 in a targeted manner, i.e. in particular in a user-specific manner, for example in order to achieve the best possible effect of the orthosis 1.

Consequently, the structural properties of the orthosis 1 can generally be adjusted or changed in a targeted manner, i.e. in particular user-specifically, via the number or arrangement or orientation of various strut elements 4a-4x, for example in order to achieve the best possible effect of the orthosis 1. In this context, as shown by way of example in FIGS. 13-18, it can be expedient to provide a plurality of receiving or fastening areas 2a, 3a on the first orthosis part 2 and/or on the second orthosis part 3, for example medially and/or laterally, so that the first and/or the second orthosis part 2, 3, in particular medially and/or laterally, is basically prepared for a plurality of strut elements 4a-4x.

Furthermore, strut elements 4a-4x can be used, which differ in their structural properties, i.e. especially in their stiffness.

The configuration of the orthosis 1 according to the exemplary embodiment shown in FIGS. 17 and 18 in two different perspective views shows that the orthosis 1 can comprise a stabilizing element 8, i.e. in particular an ankle and/or lower leg stabilizing element, ankle stabilizing element, for stabilizing an ankle area of a user. A corresponding stabilizing element 8 can be arranged or formed on respective strut elements 4a-4x, for example. In particular, a corresponding stabilizing element 8 can be penetrated by various strut elements 4a-4x, which ensures a particularly stable arrangement. Specifically, a corresponding stabilizing element 8 can be formed from a plurality of, for example half-shell-like, stabilizing element segments 8a, 8b, which are connected to each other in a form-fitting and/or frictionally engaging and/or integrally bonded manner. In the exemplary embodiment according to FIGS. 17 and 18, the stabilizing element segments 8a, 8b are connected to each other by frictional engagement via screw connections.

At least one interface 8.1 can also be arranged or formed on a stabilizing element 8 for fastening a fixing means, in particular a band-like or band-shaped fixing means. A fixing means connected to a corresponding interface 8.1 can, if necessary, additionally, allow a stabilization of the ankle and/or lower leg of a user.

Individual, a plurality of, or all features described in conjunction with an exemplary embodiment can be combined with individual, a plurality of, or all features of at least one further exemplary embodiment.

1. An orthosis for a lower extremity of a wearer, in particular for a foot and a lower leg of a wearer, preferably for wearers with a diabetic foot syndrome or a neuropathic osteoarthropathy relating to the foot, the orthosis comprising:
a first orthosis part which can be arranged or which is to be arranged on a lower leg of a wearer,
a second orthosis part which can be arranged or which is to be arranged on a foot of a wearer, and
a third orthosis part connecting the first and second orthosis part to one another, wherein
at least one of the orthosis parts comprises, at least in portions, possibly completely, a geometric-constructive design which is individually configured on the basis of data describing a foot and/or a lower leg of a wearer.

2. (canceled)

3. The orthosis according to claim 1, wherein the first orthosis part comprises a main body which comprises a receiving region, in particular accessible via an access region, for at least partially receiving a lower leg portion of a wearer.

4. The orthosis according to claim 3, wherein at least one, in particular tab-like or -shaped, interface for fastening a, in particular band-like or -shaped, fixing means for fixing, in particular as required, a lower leg portion of a wearer in the receiving region is arranged or formed on the main body.

5. The orthosis according to claim 3, wherein at least one, in particular hole-like or -shaped, interface for the fastening of an, in particular, form-fitting and/or frictionally engaging, fastening means for fastening, in particular releasably, the third orthosis part to the first orthosis part is arranged or formed on the main body.

6. The orthosis according to claim 1, wherein the second orthosis part comprises a main body which has a receiving region, in particular accessible via an access region, for at least partially receiving a foot of a wearer.

7. The orthosis according to claim 5, wherein at least one, in particular tab-like or -shaped, interface for fastening a, in particular band-like or -shaped, fixing means for fixing, in particular as required, a lower leg portion of a wearer in the receiving region is arranged or formed on the main body.

8. The orthosis according to claim 6, wherein at least one, in particular hole-like or -shaped, interface for fastening a fastening means, in particular acting in a form-fitting and/or frictionally engaging manner, for fastening, in particular releasably, the third orthosis part to the second orthosis part is arranged or formed on the main body.

9. The orthosis according to claim 1, wherein the third orthosis part has an elongate, in particular rail- or strut-like, geometric-constructive shape.

10. The orthosis according claim 1, wherein the third orthosis part comprises one or more stiffening elements extending in the longitudinal direction.

11. The orthosis according to claim 1, wherein at least two orthosis parts are movable relative to each other in at least one degree of freedom of movement.

12. The orthosis according to claim 1, wherein at least one interface for the, in particular detachable, fastening of a sole element is arranged or formed from the second orthosis part.

13. The orthosis according to claim 1, wherein the third orthosis part is arranged or formed to the side, in particular medially or laterally with respect to the condition of the orthosis when worn as intended.

14. The orthosis according to claim 1, wherein the third orthosis part is formed by one or more strut elements or comprises one or more strut elements.

15. The orthosis according to claim 14, wherein one or more strut elements is/are arranged or formed medially, in particular when the orthosis is worn as intended, and/or one or more strut elements is/are arranged or formed laterally, in particular when the orthosis is worn as intended.

16. The orthosis according to claim 1, further comprising an ankle stabilizing element which is or can be arranged in the region of an ankle of a user, in particular when the orthosis is worn as intended.

17. A method for manufacturing at least one orthosis part for an orthosis for a lower extremity of a wearer, in particular for a foot and a leg of a wearer, preferably for wearers with diabetic foot syndrome, further comprising the steps of:
generating or providing wearer data describing a foot and/or a lower leg of a wearer of the orthosis to be produced,
manufacturing at least one orthosis part on the basis of the generated or provided data.

18. (canceled)

19. The method according to claim 17, wherein the wearer data, optionally classified according to specific features or feature categories, are stored in a data storage facility.

20. The method according to one of the preceding claim 17, further comprising
providing a plurality of data describing a preconfigured geometric-constructive design of an orthosis part in a data storage facility,
selecting data describing a preconfigured geometric-constructive design of a specific orthosis part on the basis of the provided wearer data from the data storage facility,
modifying the data on the basis of the provided wearer data to generate modified data,
manufacturing the orthosis part on the basis of the modified data.

21. The method according to claim 20, further comprising adapting data describing the preconfigured geometric-constructive design of an orthosis part on the basis of the provided wearer data in order to generate construction data relating to the geometric-constructive design of the orthosis part to be produced.

22. The method according to one of claim 17, wherein the construction data describe the orthosis part to be produced with a negative offset, in particular with respect to a support element to be fastened to the orthosis part.
